(19) United States
(12) Patent Application Publication
(10) Pub. No.: US 2022/0349890 A1
(43) Pub. Date: Nov. 3, 2022

(54) A METHOD FOR PREDICTING RISK OF RECURRENCE FOR EARLY-STAGE COLON CANCER BY MEASURING FOCAL ADHESION KINASE

(71) Applicant: ARIZONA BOARD OF REGENTS ON BEHALF OF THE UNIVERSITY OF ARIZONA, Tucson, AZ (US)

(72) Inventor: William G. Cance, Phoenix, AZ (US)

(21) Appl. No.: 17/621,123

(22) PCT Filed: Jun. 18, 2020
(86) PCT No.: PCT/US20/38443

§ 371 Date: Dec. 20, 2021

Related U.S. Application Data
(60) Provisional application No. 62/862,862, filed on Jun. 18, 2019.

(51) Int. Cl. G01N 33/574; G01N 33/68

(52) U.S. Cl. CPC G01N 33/57419; G01N 33/68

(57) ABSTRACT

The present invention relates to a method of using Focal Adhesion Kinase (FAX) as a predictive marker to identify patients with early-stage colorectal cancer (CRC) at risk for recurrence. In particular, the present invention is a prognostic assay using FAK protein expression to predict those CRC patients with Stage I disease who may recur. The present invention will allow for quantitative measurement of the expression of FAK in tumor tissue of patients with early-stage CRC. As patients with stage I CRC are typically only treated with surgical resection, the present invention will uniquely facilitate the identification of those early-stage CRC patients who are at risk of recurrence and who may benefit from adjuvant therapy after resection of the primary cancer. This invention is not limited to patients with CRC and may be utilized for any condition that is caused by or causes FAK overexpression or dysfunction.

### CROSS-REFERENCES TO RELATED APPLICATIONS

This application claims benefit of U.S. Provisional Application No. 62/862,862 filed Jun. 18, 2019 the specification(s) of which is/are incorporated herein in their entirety by reference.

### STATEMENT REGARDING FEDERALLY SPONSORED RESEARCH OR DEVELOPMENT

This invention was made with government support under Grant No. R01 CA065910 awarded by the National institutes of Health. The government has certain rights in the invention.

### FIELD OF THE INVENTION

The present invention relates to a method for using Focal Adhesion Kinase (FAK) as a predictive marker for risk of recurrence, patients with Stage I colorectal cancer (CRC). In particular, the present invention is a prognostic assay using primary tumor FAK protein overexpression to predict those Stage I CRC patients at risk for recurrence who would benefit from additional therapy or enhanced monitoring

### BACKGROUND OF THE INVENTION

A major challenge in the care of the colorectal cancer (CRC) patient is predicting which early-stage patients have a high likelihood of recurrence. Up of 10% of Stage I patients experience disease recurrence. Despite these significant recurrence rates, studies have not shown benefit from adjuvant chemotherapy (e.g., adjuvant chemotherapy indicates chemotherapy after resection) for Stage I disease. At the same time, National Comprehensive Cancer Network (NCCN) guidelines recommend minimal surveillance for many of these patients. Currently there are no clinical or tumor features that predict benefit from either enhanced surveillance or adjuvant chemotherapy.

One of the most provocative yet enigmatic biomarkers in cancer is Focal Adhesion Kinase (FAK.) This protein tyrosine kinase overexpressed in most solid tumors, providing a diversity of signals that promote tumor invasion, metastasis, and immune suppression. FAK integrates signals from other receptor tyrosine kinases as well as the tumor microenvironment to protect tumor cell survival during the complex processes involved in growth, invasion, and metastasis. This myriad of complex signaling events highlights the importance of developing therapeutics that target FAK as well as appropriate correlative biomarkers.

Overexpression of FAK in CRC has been previously shown using mRNA analyses. An antibody that recognizes FAK in formalin-fixed, paraffin-embedded tissues was developed and used in small studies of FAK expression in CRC, demonstrating minimal expression in normal tissue and increased expression in primary tumors. Though, it is unclear to what extent the level of FAK predicts a more aggressive biological behavior for a given tumor. A meta-analysis of FAK in solid tumors found that FAK is associated with worse overall survival (OS) in many tumor types. However, in subgroup analysis, there was no association between high FAK and outcomes in CRC. Individual studies of FAK in CRC have conflicting results, with some showing positive association between high FAK and poor prognosis, while others finding no association.

A common feature of most studies of FAK in colorectal cancer was their inclusion of only small numbers of early stage patients. These studies also did not consistently correlate FAK with primary tumor clinicopathologic features, with infrequent exceptions. Finally, to further complicate this problem, these studies used a wide range of antibodies for the immunohistochemical analyses of FAK, with varying levels of sensitivity for detecting FAK, and targeting various protein functions of FAK. While results from some studies demonstrated FAK is highly expressed in CRC, the prognostic implications of high FAK in this disease remain unclear.

The present invention features a method of using primary tumor FAK overexpression to predict risk of recurrence of Stage I CRC. The method features a clinically adaptable immunohistochemical scoring system for FAK in colorectal cancer based on a simplified high FAK vs. low FAK readout. The method uses a monoclonal anti-FAK antibody (e.g., MAb 4.47) specifically created to detect FAK in formalin-fixed, paraffin-embedded tissue sections. Without wishing to limit the present invention to any theory or mechanism, it is believed that high levels of FAK (or overexpression of FAK) in Stage I primary CRC, but not Stage II, III, or IV CRC, are associated with a worse prognosis and identify a subset of early-stage CRC patients who are at high risk of recurring and who would benefit from additional therapy (e.g., vaccine therapy, adjuvant chemotherapy, adjuvant immunotherapy, adjuvant radiation, radical surgical resection) and/or more rigorous surveillance protocols.

### BRIEF SUMMARY OF THE INVENTION

It is an objective of the present invention to provide methods of using tumor FAK overexpression that allow for predicting risk of recurrence in subjects with Stage I CRC, identifying a subset of subjects with early-stage CRC who would benefit from additional therapy or enhanced surveillance, and treating subjects with Stage I CRC as specified in the independent claims. Embodiments of the invention are given in the dependent claims. Embodiments of the present invention can be freely combined with each other if they are not mutually exclusive.

One of the unique and inventive technical features of the present invention is the use of Stage I tumor FAK overexpression as a prognostic indicator and early predictor of risk of recurrence in patients with Stage I CRC. Without wishing to limit the invention to any theory or mechanism, it is believed that FAK overexpression observed in select tumors advantageously provides for identifying early a subset of patients with Stage I CRC who have a high risk of recurrence and who would benefit from additional therapy. None of the presently known prior references or work have successfully demonstrated FAK expression in CRC to be predictive of recurrence risk. Furthermore, the prior references teach away from the present invention. For example, the prior art teaches that while FAK is overexpressed in CRC, this FAK overexpression is not consistently prognostic in CRC and that the FAK overexpression is similar between primary and metastatic CRC tumors as compared to that observed in normal colon tissue. Because only 10% of Stage I CRC recurs, while all the metastatic tumors are recurrences, it would not be obvious to use the FAK overexpression observed in primary CRC tissue, which has similar FAK expression to that found in metastatic tumors, as a predictor of recurrence for Stage I CRC. In contrast to the previously reported lack of or inconsistent FAK prognostic significance in early-stage CRC, the present invention features a method of using FAK overexpression observed in select Stage I tumors as an independent prognostic marker to predict recurrence risk for a subset of subjects with Stage I CRC. For example, FAK expression in Stage I CRC tumors predicted higher risk of recurrence, while FAK overexpression in more advanced tumors did not predict recurrence. Without wishing to limit the present invention to any theory or mechanism, FAK overexpression in tumors from select Stage I subjects surprisingly identified a subset of patients with CRC at high risk for recurrence and who may benefit from additional treatment and monitoring, including not limited to more aggressive surveillance for recurrent CRC, more aggressive surgery (i.e., radical rectal resection rather than local excision), adjuvant therapy (including but not limited to chemotherapy, immunotherapy, and radiation therapy), or vaccine therapy, For example, high FAK expression in a rectal cancer may indicate a need for either adjuvant radiation therapy and/or radical rectal resection contradicting prior art teaching of local excision as sole therapy. Conversely, low FAK expression in a rectal cancer would be a potential indication for local excision alone and obviate the need for radiation therapy and/or radical rectal resection. Other advantages may become apparent to one skilled in the art upon practice of the embodiments described and enabled herein.

In general, the present invention features methods for treating and predicting a risk, of recurrence for Stage I CRC, identifying a subject with Stage I CRC at risk for recurrence who would benefit from additional therapy or enhanced monitoring, preventing CRC recurrence in a subject with early-stage CRC, prognosing the subject with early-stage CRC, treating the subject with early-stage CRC, and monitoring the treatment and CRC progression in a subject with early-stage CRC. These methods use FAK protein expression and/or overexpression observed in Stage I primary tumor tissue. In preferred embodiments, risk profiles and clinical scores are determined and are based on the expression or overexpression of FAK detected in Stage I tumors. For example, a clinical score of 0 indicates no detectable FAK expression, a clinical score of 1 indicates low FAK expression, a clinical score of 2 indicates moderate FAK expression, and a clinical score of 3 indicates high FAK expression. Typically, those subjects with a clinical score of 0 or 1 would have a low risk of recurrence and may not benefit (considering risk-benefit ratio) from adjuvant therapy. While subjects with a clinical score of 2 or 3 would have a higher risk of recurrence and may benefit from adjuvant therapy.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1A shows a representative immunohistochemistry (IHC) image of a colorectal primary tumor TMA core scoring 0 (no staining).

FIG. 1B shows a representative IHC image of a colorectal primary tumor TMA core scoring 1 (weak staining).

FIG. 1C shows a representative IHC image of a colorectal primary tumor TMA core scoring 2 (moderate staining).

FIG. 1D shows a representative IHC image of a colorectal primary tumor TMA core scoring 3 (strong staining). Patient samples were dichotomized as “low FAK expression” if scored a 0 or 1, and “high FAK expression” if scored a 2 or 3.

FIGS. 2A-2C show Kaplan-Meier curves for overall survival by FAK expression in primary colorectal adenocarcinoma. Initial cohort (FIG. 2A), and validation cohort (FIG. 2B). High FAK defined as moderate to strong staining, and low FAK defined as no or weak staining. High FAK expression in primary colorectal adenocarcinoma predicts worse clinical outcome. *Totals based on available clinical data.

FIGS. 3A-3G show Kaplan-Meier curves for overall survival, disease specific survival, and recurrence-free survival, by FAK expression in primary colorectal adenocarcinoma. Initial cohort (FIGS. 3A-3C), and validation cohort (FIGS. 3D-3F) show similar outcomes for overall survival rate (FIG. 3A and FIG. 3D), disease specific survival rate (FIG. 3B and 3E) and recurrence free rate (FIG. 3C and FIG. 3F). High FAK expression in primary colorectal adenocarcinoma predicts worse clinical outcome. *Totals based on available clinical data.

### DETAILED DESCRIPTION OF THE INVENTION

All patents, applications, published applications and other publications are incorporated by reference in their entirety and for all purposes. Unless defined otherwise, all technical and scientific terms used herein have the same meaning as commonly understood by those of ordinary skill in the art to which the invention belongs.

Any methods, devices and materials similar or equivalent to those described herein can be used in the practice of this invention. The following definitions are provided to facilitate understanding of certain terms used frequently herein and are not meant to limit the scope of the present disclosure. In the event that there is a plurality of definitions for a term herein, those in this section prevail unless stated otherwise. Headings used herein are for organizational purposes only and in no way limit the invention described herein.

As used herein, the term “overexpression” refers to an abnormal or artificial expression of a molecule (e.g., protein, mRNA, DNA) with increased quantity. For example, a higher expression of FAK in a tumor tissue sample as compared to that in paired normal tissue indicates that FAK is overexpressed in the tumor tissue.

As used herein, “administering” and the like refer to the act physically delivering a composition or other therapy (e.g. an immunotherapy) described herein into a subject by such routes as oral, mucosal, topical, transdermal, suppository, intravenous, parenteral, intraperitoneal, intramuscular, intralesional, intrathecal, intranasal or subcutaneous administration. Parenteral administration includes intravenous, intramuscular, intra-arterial, intradermal, subcutaneous, intraperitoneal, intraventricular, and intracranial administration. When a disease, disorder or condition, or a symptom thereof, is being treated, administration of the substance typically occurs after the onset of disease, disorder or condition or symptoms thereof. When a disease, disorder or condition, or symptoms thereof, are being prevented, administration of the substance typically occurs before the onset of the disease, disorder or condition or symptoms thereof.

As used herein, the terms “subject” and “patient” are used interchangeably. As used herein, a subject can be a mammal such as a non-primate (e.g., cows, pigs, horses, cats, dogs, rats, etc.) or a primate (e.g., monkey and human). In specific embodiments, the subject is a human. In one embodiment, the subject is a mammal (e.g., a human) having a disease, disorder or condition described herein. In another embodiment, the subject is a mammal (e.g., a human) at risk of developing a disease, disorder or condition described herein. In certain instances, the term patient refers to a human.

The terms “treating” or “treatment” refer to any indicia of success or amelioration of the progression, severity, and/or duration of a disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating: or improving a patient's physical or mental well-being. As described herein, “additional treatment” refers to aggressive surgery (i.e., radical rectal resection rather than local excision), adjuvant therapy (including but not limited to chemotherapy, immunotherapy, and radiation therapy), or vaccine therapy. Adjuvant therapy is typically administered after resection of the primary tumor with no gross evidence of residual cancer. Other non-limiting examples of adjuvant therapy comprise inhibitors to the FAK pathway, which result in decrease production, expression, and/or activity of FAK, antibodies, vaccines, small molecules, cytotoxic agents (e.g. 5-fluorouracil, leucovorin, oxaliplatin; FOLFOX), tyrosine kinase inhibitors including both small molecules and antibody-based inhibitors (e.g., cetuximab), immunotherapy including but not limited to PD-1 and PDL-1 inhibitors such as atezolizumab and pembrolizumab, additional surgical therapy, and/or additional radiation therapy.

The terms “manage,” “managing,” and “management” refer to preventing or slowing the progression, spread or worsening of a disease or disorder, or of one or more symptoms thereof. In certain cases, the beneficial effects that a subject derives from a prophylactic or therapeutic agent do not result in a cure of the disease or disorder.

The term “preventing” refers to the treatment with or administration of an agent provided herein, with or without other additional active agent (e.g. an immunotherapy for cancer), prior to the onset of symptoms, particularly to patients at risk of cancer and/or other disorders described herein. The term also refers to coadministration of a treatment (e.g., anti-FAK therapy) with other therapies including standard-of-care therapies for cancer (e.g., immunotherapy) as described herein. It should be understood that the treatments described herein can be co-administered with one or more standard-of-care therapies for cancer described herein. The term prevention includes the inhibition or reduction of a symptom of the particular disease, as well as a reduced incidence of a symptom of the particular disease (e.g. by comparison to historical data for a given subject, or population data for similar subjects). Patients with familial history of a disease in particular are candidates for preventive regimens in certain embodiments. In addition, patients who have a history of recurring symptoms are also potential candidates for the prevention. In this regard, the term “prevention” may be interchangeably used with the term “prophylactic treatment.”

A prophylactically effective amount of an agent (e.g. a cytotoxic agent, immunotherapy) means an amount of therapeutic agent, alone or in combination with other agents, which provides a prophylactic benefit in the inhibition or reduced incidence of a symptom of a disease or recurrence of a disease. The term also refers to coadministration of a treatment (e.g., anti-FAK therapy) with other therapies including standard-of-care therapies for cancer (e.g., chemotherapy, immunotherapy). The term prophylactically effective amount can encompass an amount that improves overall prophylaxis or enhances the prophylactic efficacy of another prophylactic agent.

The term “effective amount” as used herein refers to the amount of a therapy or intervention which is sufficient to reduce and/or ameliorate the severity and/or duration of a given disease, disorder or condition and/or a symptom related thereto. This term also encompasses an amount necessary for the reduction or amelioration of the advancement or progression of a given disease, disorder or condition, reduction or amelioration of the recurrence, development or onset of a given disease, disorder or condition, and/or to improve or enhance the prophylactic or therapeutic effect(s) of another therapy. In some embodiments, “effective amount” as used herein also refers to the amount of therapy provided herein to achieve a specified result.

As used herein, and unless otherwise specified, the term “therapeutically effective amount” of an agent described herein, or a standard-of-care treatment described herein, is an amount sufficient to provide a therapeutic benefit in the treatment or management of a cancer, or to delay or minimize one or more symptoms associated with the presence of the cancer. A therapeutically effective amount of an agent described herein or a standard-of-care therapy for cancer described herein means an amount of therapeutic agent, alone or in combination with other therapies, which provides a therapeutic benefit in the treatment or management of the cancer. The term “therapeutically effective amount” can encompass an amount that improves overall therapy, reduces or avoids symptoms or causes of cancer, or enhances the therapeutic efficacy of another therapeutic agent.

A therapy is any protocol, method and/or agent that can be used in prevention, management, treatment and/or amelioration of a given disease, disorder or condition. In certain embodiments, the terms “therapies” and “therapy” refer to a drug therapy, biological therapy, supportive therapy, radiation therapy, physical therapy, vaccine therapy, and/or other therapies useful in the prevention, management, treatment and/or amelioration of a given disease, disorder or condition known to one of skill in the art such as medical personnel.

A regimen is a protocol for dosing and timing the administration of one or more therapies (e.g., combinations described herein, another active agent such as for example an anti-proliferative agent described herein, or other therapy for cancer described herein) for treating a disease, disorder, or condition described herein. A regimen can include periods of active administration and periods of rest as known in the art. Active administration periods include administration of combinations and compositions described herein and the duration of time of efficacy of such combinations, compositions, and physical therapies. Rest periods of regimens described herein include a period of time in which no agent is actively administered, and in certain instances, includes time periods where the efficacy of such agents can be minimal. Rest periods of regimens described herein can include a period of time in which no physical therapy is actively performed. Combination of active administration and rest in regimens described herein can increase the efficacy and/or duration of administration of the combinations and compositions described herein.

As used herein, the term “Companion Diagnostic (CDx)” assays, as defined by the FDA, are in vitro diagnostics (IVD) devices that provide information essential for the safe and effective use of a corresponding therapeutic product. The FDA specifies three main areas where a CDx assay is essential: 1) Identify patients who are most likely to benefit from a particular therapeutic product; 2) identify patients likely to be at increased risk of serious adverse reactions as a result of treatment with a particular therapeutic product; and 3) To monitor response to treatment for the purpose of adjusting treatment (e.g., schedule, dose, discontinuation) and to achieve improved safety or effectiveness. A CDx can be used both to predict outcome (efficacy and safety) and to monitor response. The present invention also may be used a CDx for CRC vaccines, defining those subjects (e.g., early-stage patients with a higher risk of recurrence, with FAK clinical scores of 2 or 3) who would receive CRC vaccines.

As used herein, the term “prognosis” is used for predicting the likely or expected development of a disease, including whether the signs and symptoms will improve or worsen or remain stable over time; expectations of quality of life, such as the ability to carry out daily activities; the potential for complications and associated health issues; and the likelihood of survival.

As used herein, the term “diagnosis” is used to define the process of determining which disease or condition explains a person's symptoms and signs.

As used herein, the term “well differentiated” is used to define cancer cells that are organize and have some features of normal tissue. As used herein, the term “poorly differentiated” is used to define cancer cells that don't look like normal cells and appear disorganized under the microscope and tend to grow and spread quickly. Cancer cells that do not fit these criteria are called moderately differentiated.

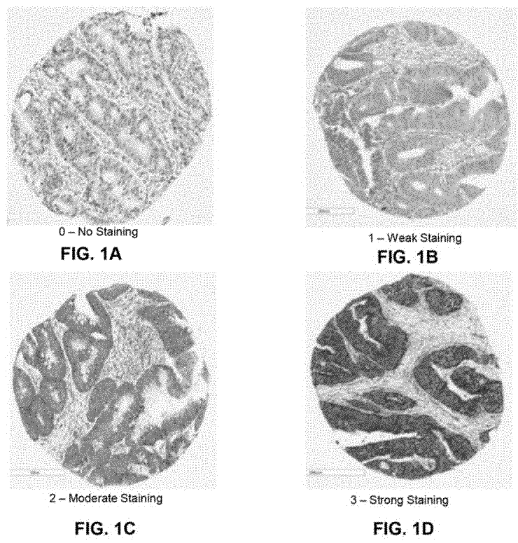

Referring now to FIGS. 1-3, the present invention features methods of using FAK overexpression that allow for predicting risk of recurrence of Stage I CRC, determining therapeutic approaches for subjects with Stage I CRC, identifying a subset of subjects with Stage I CRC who would benefit from adjuvant therapy, and treating subjects with Stage I CRC.

The present invention features a method of preparing a tissue sample from a subject afflicted with Stage I CRC. In some embodiments, the method comprises first obtaining a tissue sample from a subject with Stage I CRC. Next, the tissue samples are separated by measuring the focal adhesion kinase (FAK) protein expression in the tissue sample using immunohistochemical staining. The clinical score of FAK protein expression is then derived based on the level of immunohistochemical staining. For example, no FAK staining comprises no detectable FAK protein expression and represents a clinical score of 0. Weak FAK staining comprises low FAK protein expression and represents a clinical score of 1, moderate FAK staining comprises moderate FAK protein expression and represents a clinical score of 2, and strong FAK staining comprises high FAK expression and represents a clinical score of 3. The clinical score of FAK protein expression is then analysed.

The present invention also features a method for personalized treatment of a subject afflicted with Stage I. In some embodiments, the methods comprise determining whether the subject has an overexpression of focal adhesion kinase (FAK). Overexpression of FAK is determined by first obtaining a tissue sample from the subject that is amenable to measure FAK protein expression. The FAK protein expression is then measured in the tissue sample using immunohistochemical staining. The clinical score of FAK protein expression is then derived based on the level of immunohistochemical staining. For example, no FAK staining comprises no detectable FAK protein expression and represents a clinical score of 0. Weak FAK staining comprises low FAK protein expression and represents a clinical score of 1, moderate FAK staining comprises moderate FAK protein expression and represents a clinical score of 2, and strong FAK staining comprises high FAK expression and represents a clinical score of 3. A therapeutically effective drug or other intervention(s) is then administered to treat the early-stage CRC based on the pre-classified clinical score and associated risk of recurrence. For example, clinical scores of 0 and 1 predict low risk of recurrence and clinical scores of 2 and 3 predict higher risk of recurrence of cancer in subjects with early-stage CRC. Therefore, those subjects with clinical scores of 0 or 1 with low risk of recurrence would not be administered a therapeutic drug or intervention and those subjects with clinical scores of 2 or 3 with higher risk of recurrence would be administered a therapeutic drug or intervention to treat the Stage I CRC.

The present invention features a method for predicting a risk of recurrence of cancer in subjects with Stage I CRC using clinical scores based on the expression or overexpression of FAK detected in Stage I tumors. In preferred embodiments, clinical scores are based on the FAK protein expression in primary tumor tissue as measured by immunohistochemistry. In some embodiments, the method comprises first determining whether the subject has an overexpression of FAK by obtaining a tissue sample from the subject that is amenable to measure FAK protein expression. The FAK protein expression is then measured in the tumor tissue sample using immunohistochemical staining. The clinical score of FAK protein expression is then derived based on the level of immunohistochemical staining. For example, no FAK staining comprises no detectable FAK protein expression and represents a clinical score of 0. Weak FAK staining comprises low FAK protein expression and represents a clinical score of 1, moderate FAK staining comprises moderate FAK protein expression and represents a clinical score of 2, and strong FAK staining comprises high FAK expression and represents a clinical score of 3. The risk of recurrence is then determined based on the previously classified clinical score. For example, the clinical scores of 0 and 1 would predict low (or lower) risk (and not predict a high risk) of recurrence of cancer in subjects with early-stage CRC who then are called or considered to have low-risk, early-stage CRC. The clinical scores of 2 and 3 would predict higher risk of recurrence of cancer in subjects with Stage I CRC who then are called or considered to have high-risk Stage I CRC.

As used herein “Immunohistochemistry (IHC)” refers to a microscopy-based technique for visualizing cellular components, such as proteins or other macromolecules in tissue samples. The strength of IHC is the intuitive visual output that reveals the presence and localization of the target-protein in the context of different cell types, biological states, and/or subcellular localization within tissue samples. In some embodiment, IHC is able detect not only whether a protein is present but also reveal the relative quantity of the protein in a tissue sample based on the amount of staining. In some embodiment, an increase in IHC staining may indicate that a protein that is overexpressed.

The IHC scoring systems of 0-3 is standard practice in pathology and is well known in the art such that it is extensively used to visualize the expression level protein. Non-limiting examples can be found in FIGS. 1A-1D wherein FIG. 1A represents a score of 0 (no expression), FIG. 1B represents a score of 1 (low expression), FIG. 1C represents a score of 2 (moderate expression) and FIG. 1D represents a score of 3 (high expression). Additionally, the same IHC scoring system of 0-3 is regularly used to determine human epidermal growth factor receptor (HER2) expression, as well as the expression of estrogen receptor (ER) and progesterone receptor (PR) in Breast Cancer.

In some embodiments, the sample of tissue comprises biopsy tissue and/or surgical resection tissue. These tissues may be formalin-fixed, paraffin-embedded tissue or frozen tissue. Non-limiting examples of tissue samples comprise tumor tissue, benign tissue, normal tissue, fibrotic tissue, and/or tissue culture.

In some embodiments, the method features a biological sample comprising tissue, blood, or cells. Non-limiting examples of tissue comprises frozen tissue, formalin-fixed, paraffin-embedded tissue, tumor tissue, non-tumor tissue, benign tissue, fibrotic tissue, and normal tissue. In other embodiments, the cell sample comprises cell culture or circulating tumor cells. In other embodiments, the blood sample comprises, serum, plasma, peripheral blood mononuclear cells, circulating tumor cells, circulating tumor DNA, circulating free DNA, circulating microRNA.

In preferred embodiments, FAK protein expression is determined using a monoclonal antibody, M4.47. In other embodiments, the FAK protein expression in a tissue sample is furthered measured by standard technologies for protein analysis comprising immunofluorescence, Western blot, and/or ELISA. In some embodiments, the method may feature examining FAK expression at the FAK mRNA level, including but not limited to whole transcriptome analyses and/or a FAK activity/phosphorylation level, using standard genomic and proteomic techniques, including but not limited to phosphorylation analyses of tyrosine residues of FAK. In other embodiments, FAK may also be measured in blood/serum/plasma assays.

In some embodiments, the method uses FAK expression comprising FAK protein levels, FAK DNA levels, FAK mRNA levels, and/or FAK activity levels. The FAK expression is measured by standard analytic techniques comprising immunohistochemistry, immunochemistry, immunofluorescence, ELISA, Western blot, Northern blot, reverse transcription/polymerase chain reaction, whole transcriptome sequencing (WTS), and/or measurement of phosphorylation status of tyrosine residues on FAK (e.g., Y397, Y925 and others).

In some embodiments and when samples can be obtained throughout time (e.g., blood samples or other fairly non-invasive means), the FAK expression can be measured longitudinally, at various times throughout the progression of the cancer. Non-limiting examples comprise measuring FAK expression at: 1) at time of diagnosis; 2) two to seven days post-diagnosis, 3) one-month post-diagnosis, 4) three months post-diagnosis, or 5) >three months post-diagnosis.

In preferred embodiments, the early-stage colorectal cancer is Stage I or Stage II CRC. In some embodiments, the method is for further assessing FAK expression in blood and/or cell or tissue culture. For example, FAK expression may be assessed in circulating tumor cells or circulating DNA.

In other preferred embodiments, the method is to prolong life. Non-limiting examples comprise prolonging life by at least about 1 day, by at least about 1 week, by at least about 1 month, by at least about 6 months, or by at least about 12 months. In some embodiments, the method is for personalizing initiation and continuation of therapy.

In preferred embodiments, the method is for subjects with early-stage cancer comprising Stage I cancer or Stage II cancer. Non-limiting examples early-stage cancer comprise Stage I colon cancer, Stage II colon cancer, Stage I CRC, Stage II CRC, low-grade and/or early-stage I or II lung cancer, liver cancer, melanoma, breast cancer, prostate cancer, thyroid cancer, ovarian cancer, anal cancer, cervical cancer, sarcoma, pancreatic cancer, and/or cholangiocarcinoma. In other embodiments, the method also could be used to detect severity of disease and cancer risk from fibrotic diseases such as cirrhosis of the liver and pulmonary fibrosis.

In other embodiments, the method is for longitudinal assessment of cancer (e.g., CRC) progression and prognosis. A non-limiting example comprises a longitudinal assessment with more frequent screening for patients with early-stage Stage I or II CRC and FAK clinical scores of 2 or 3. Non-limiting examples of screening comprise measurements of CEA, CT scans, MRI, and/or PET-CT scans. In some embodiments, the screening is performed at least weekly, at least bi-weekly, at least monthly, at least bi-monthly, at least yearly, or at least bi-yearly.

In some embodiments, no FAK staining comprises no detectable FAK protein expression and represents a clinical score of 0. Weak FAK staining comprises low FAK protein expression and represents a clinical score of 1, moderate FAK staining comprises moderate FAK protein expression and represents a clinical score of 2, and strong FAK staining comprises high FAK expression and represents a clinical score of 3. The risk of recurrence profile is then determined based on the previously classified clinical score. For example, the clinical scores of 0 and 1 would not predict a high risk of recurrence cancer in subjects with early-stage cancer and the clinical scores of 2 and 3 would predict higher risk of recurrence of cancer in subjects with early-stage cancer. Non-limiting examples comprise the subjects at low risk of recurrence with clinical scores of 0 and 1 would not benefit from adjuvant therapy and those subjects at higher risk for recurrence with clinical scores of 2 and 3 would benefit from additional therapy. The subjects with early-stage cancer at risk of recurrence may then benefit from additional therapy. Non-limiting examples of additional therapy or intervention comprise aggressive or radical surgical resection, radiation therapy, vaccine therapy, antibodies, and/or adjuvant chemotherapy, immunotherapy, and/or radiotherapy. These subjects with early-stage cancer at risk of recurrence may also benefit from more frequent monitoring or screening. Non-limiting examples of screening tests for CRC comprise serum CEA levels, CT scans (e.g., of abdomen, pelvis, chest), MRI scans, and/or PET-CT scans.

In some embodiment, subjects with clinical scores of 0 or 1 with low risk of recurrence would not require a therapeutic drug or intervention to prevent recurrence and those subjects with clinical scores of 2 or 3 with higher risk of recurrence would require a therapeutic drug or intervention to prevent recurrence. Non-limiting examples of therapeutic drug or intervention comprises screening tests for CRC that may include serum carcinoembryonic antigen (CEA) levels, computed tomography (CT) scans, magnetic resonance imaging (MRI) scans, and/or positron-emission tomography-computed tomography (PET-CT) scans; vaccine therapy, additional radical surgery, radiation therapy, chemotherapy, and/or immunotherapy. Additional non-limiting examples of interventions include, adjuvant therapy (i.e., therapy after surgical resection of primary tumor) comprising of inhibitors to the FAK pathway, which result in decrease production, expression, and/or activity of FAK, or antibodies, vaccines, small molecules, cytotoxic agents, additional surgical therapy, immunotherapy, chemotherapy, and/or radiation therapy.

### EXAMPLE

The following is a non-limiting example of the present invention. It is to be understood that said example is not intended to limit the present invention in any way. Equivalents or substitutes are within the scope of the present invention.

Initial cohort tumor Specimens: Tissue samples from surgical specimens were from collected from 298 patients with CRC treated at Roswell Park Cancer Institute (RPCI) between 1993-2011. One hundred seventy-seven (59.4%) primaries were colon and 121 (40.6%) rectal origin. Tissue microarrays (TMAs) consisting of 0.6 mm cores per sample were created from formalin-fixed, paraffin-embedded (FFPE) tissue blocks. Matching normal colonic tissue was obtained in 290 of these patients, and 47 patients had matching metastatic tissue. To address potential tissue heterogeneity, samples were obtained in triplicate from surgical specimens.

Immunohistochemical Analyses: The slides were stained with FAK 4.47 antibody (Millipore), developed for use in immunohistochemical analysis of FFPE specimens (Cance W G, Harriss J E, Iacocca M V, et al. Immunohistochemical Analyses of Focal Adhesion Kinase Expression in Benign and Malignant Human Breast and Colon Tissues: Correlation with Preinvasive and Invasive Phenotypes. Clin Cancer Res. 2000:6: 2417-2423). This staining protocol was modified slightly compared to our previously published studies and uses new technology that does not require a biotin-avidin reaction.

Scoring FAK expression: Cores were evaluated for FAK intensity and quantified as “0” for no staining, “1”—weak staining, “2”—moderate staining, and “3”—strong staining by a board-certified pathologist for each cohort (L. Y. and L. H. L) who were blinded to the clinical data. To characterize FAK among tissue type and clinicopathologic characteristics, the triplicate scores were averaged. To analyze FAK with outcomes data, the average scores were dichotomized into “High FAK Expression” (score 2 or 3) and “Low FAK expression” groups (score 0 or 1).

Patient characteristics and statistical analyses: Patient outcomes were assessed by chart review. Patient characteristics were summarized using the mean and standard deviation for continuous variables, and as frequencies and relative frequencies for categorical variables. Time to event outcomes were summarized using standard Kaplan-Meier methods, where estimates of the median time to event and 10-year rates were obtained with 95% confidence intervals. Overall survival (OS) was defined as time in months between diagnosis and death due to any cause or last follow-up. Recurrence-free survival (RFS) was defined as time in months between enrollment and disease recurrence, death due to, cancer, or last follow-up. Disease-specific survival (DSS) was defined as time in months between diagnosis and death due to only cancer. RFS was calculated only for patients who were disease-free after surgery.

FAK was compared between tissue sites in a pairwise fashion using Wilcoxon signed rank tests. The association between FAK (treated as continuous or dichotomous) and patient characteristics were evaluated using the Wilcoxon rank sum or Fisher's exact tests. The association between dichotomized FAK and the time-to-event outcomes were evaluated using the log-rank test. An exploratory multivariable analysis was conducted to examine the association between FAK and time-to-event outcomes while adjusting for additional clinical factors. OS, DSS, and RFS were modeled as a function of dichotomized FAK, stage, lymphovascular invasion (LVI), and perineural invasion (PNI) using Cox regression. The models were fit using Firth's method and hazard ratios with corresponding confidence intervals were obtained from model estimates. All analyses were conducted in SAS v9.4 (Cary, N C) at a significance level of 0.05.

Validation Cohort: To evaluate the findings from the initial cohort, an independent data set was examined. This validation cohort consisted only of early stage colorectal adenocarcinomas and were identified from consecutive patients who were surgically treated at Memorial Sloan Kettering Cancer Center (MSKCC) with curative intent for stage I and II CRC between 1990 and 2000 were selected. Only patients without neoadjuvant or adjuvant therapy were included, and only cases for which we had access to tissue blocks on the original resection and clinical follow-up information were included. Representative FFPE tumor blocks were selected from each of the study cases. TMAs were constructed using three random 0.6-mm cores were sampled from each tumor specimen. A total of 578 colorectal carcinomas from 578 patients were included in the TMA. Of these, 61 cases (10.6%) had tissue loss or no tumor present in the sample, resulting in 517 cases that were analyzed for FAK expression, including 47 (9.1%) from the rectosigmoid, and 470 (90.9%) from the remainder of the colon. The TMAs were stained with FAK 4.47 antibody using the same protocol and technology as the initial cohort. Cores were scored using the same scale by a second board-certified pathologist (L. H. L) who was blinded from the initial results. This pathologist was trained through images provided by the initial pathologist.

Medical record review was performed on these cases to obtain clinical data, including age, sex, recurrence, and survival, and analysis was performed as per the initial cohort. Comparisons between the initial cohort and the validation cohort was also performed.

### Development of a Clinical FAK Biomarker Test

The first objective was to develop a measure FAK expression that could be rapidly adapted for CRC specimens in the clinical setting. Using an immunohistochemical approach and scoring system of 0, 1, 2, or 3, our tumor samples were dichotomized into high FAK or low FAK subsets. High FAK expression was defined as a score of 2 or 3 and low FAK expression as a score of 0 or 1 (FIGS. 1A-1D) This approach provided a simplified scoring system that was easily adaptable to clinical practice.

### Characterization of the Patients in the Initial and Validation Cohorts

To analyze FAK as a biomarker in CRC, the present invention had a discovery cohort of patents where which was used to make initial observations. This initial cohort was comprised of primary tumor specimens from 298 patients with Stages I to IV CRC (Table 2). In this group, we observed that significantly fewer T1 and T2 tumors had high FAK expression compared to T3 tumors (Table 1). Similarly, Stage I CRC patients had fewer high FAK tumors (38.9%) than patients with stage II (61.6%), stage III (59.1%), and stage IV (60.5%) tumors (p=0.017, Table 2). The studies of FAK expression in matched normal tissues in these patients showed none to minimal expression, consistent with previous reports (data not shown). The only other significant finding in this cohort was lower FAK expression in rectal tumors compared to other sites of the colon (p=0.045, Table 2).

TABLE 1
Clinicopathologic Characteristics of Stage I Colorectal
Carcinoma of Initial and Validation Cohorts

| | | Initial Cohort | | Validation Cohort | | Overall | |
|---|---|---|---|---|---|---|---|
| | | N = 72 | | N = 185 | | N = 257 | |
| Age | <70 | 40 | (55.6%) | 99 | (53.5%) | 139 | (54.1%) |
| | >70 | 32 | (44.4%) | 86 | (46.5%) | 118 | (45.9%) |
| Age* | Mean/Std/N | 64.9/13.7/72 | | 66.7/12.4/185 | | 66.2/12.8/257 | |
| | Median/Min/Max | 67.0/27.0/98.0 | | 69.0/29.0/89.0 | | 68.0/27.0/98.0 | |
| Gender | Male | 28 | (38.9%) | 92 | (49.7%) | 120 | (46.7%) |
| | Female | 44 | (61.1%) | 93 | (50.3%) | 137 | (53.3%) |
| Location | Rectum | 38 | (52.8%) | 22 | (12.0%) | 60 | (23.4%) |
| | Colon | 34 | (47.2%) | 162 | (88.0%) | 196 | (76.6%) |
| Grade¹ | WD | | | 17 | (9.6%) | 17 | (6.9%) |
| | MD | 61 | (85.9%) | 149 | (84.2%) | 210 | (84.7%) |
| | PD | 10 | (14.1%) | 11 | (6.2%) | 21 | (8.5%) |
| T Stage² | 1 | 14 | (19.4%) | 40 | (21.6%) | 54 | (21.0%) |
| | 2 | 58 | (80.6%) | 145 | (78.4%) | 203 | (79.0%) |
| LVI | Negative | 58 | (93.5%) | 173 | (94.0%) | 231 | (93.9%) |
| | Positive | 4 | (6.5%) | 11 | (6.0%) | 15 | (6.1%) |
| PNI | Negative | 64 | (98.5%) | 179 | (98.4%) | 243 | (98.4%) |
| | Positive | 1 | (1.5%) | 3 | (1.6%) | 4 | (1.6%) |
| FAK | <2 | 44 | (61.1%) | 146 | (78.9%) | 190 | (73.9%) |
| | >=2 | 28 | (38.9%) | 39 | (21.1%) | 67 | (26.1%) |

| | | | |
|---|---|---|---|
| MMR** | Negative | 33 | (17.8%) |
| | Positive | 152 | (82.2%) |

¹WD = Well differentiated; MD = Moderately differentiated; PD = poorly differentiated
²T1 = tumor invades the submucosa; T2 = tumor invades into the muscularis propria; T3 = tumor has grown through the muscularis propria and into the subserosa
**MMR analysis was not consistently performed on the initial cohort and is thus excluded

TABLE 2
Clinicopathologic Characteristics and FAK Expression, Initial cohort

| Primary Tumor | | | Low FAK | | High FAK | | |
|---|---|---|---|---|---|---|---|
| Clinicopathologic | Patients | | (Score 0 to <2) | | (Score 2 to 3) | | |
| Characteristic | (N, %)¹ | | (N, %) | | (N, %) | | P value² |
| Age | | | | | | | 0.288 |
| <70 | 179 | (60.1%) | 85 | (47.5%) | 94 | (52.5%) | |
| >70 | 119 | (39.9%) | 49 | (41.2%) | 70 | (58.8%) | |
| Gender | | | | | | | 1.000 |
| Female | 177 | (59.4%) | 80 | (45.2%) | 97 | (54.8%) | |
| Male | 121 | (40.6%) | 54 | (44.6%) | 67 | (55.4%) | |
| Stage: | | | | | | | 0.017* |
| I | 72 | (24.2%) | 44 | (61.1%) | 26 | (38.9%) | |
| II | 99 | (33.2%) | 38 | (38.3%) | 61 | (61.6%) | |
| III | 88 | (29.5%) | 36 | (40.9%) | 52 | (59.1%) | |
| IV | 38 | (12.8%) | 15 | (39.5%) | 23 | (60.5%) | |
| T-Stage | | | | | | | 0.002* |
| 1 | 14 | (4.7%) | 8 | (57.1%) | 6 | (42.9%) | |
| 2 | 82 | (27.6%) | 49 | (59.8%) | 33 | (40.2%) | |
| 3 | 201 | (67.7%) | 76 | (37.8%) | 125 | (62.2%) | |
| Tumor Grade: | | | | | | | 0.624 |
| Low | 250 | (85.3%) | 112 | (44.8%) | 138 | (55.2%) | |
| High | 43 | (14.7%) | 21 | (48.8%) | 22 | (51.2%) | |
| Lymphovascular | | | | | | | 0.560 |
| Invasion: | | | | | | | |
| Yes | 63 | (21.1%) | 31 | (49.2%) | 32 | (50.8%) | |
| No | 200 | (67.1%) | 89 | (44.5%) | 111 | (55.5%) | |
| Perineural | | | | | | | 0.530 |
| Invasion: | | | | | | | |
| Yes | 50 | (16.8%) | 20 | (40.0%) | 30 | (60.0%) | |
| No | 235 | (78.9%) | 108 | (46.0%) | 127 | (54.0%) | |
| Location | | | | | | | 0.045* |
| Rectum | 121 | (40.6%) | 63 | (52.1%) | 58 | (47.9%) | |
| Other | 177 | (59.4%) | 71 | (40.1%) | 106 | (59.9%) | |
| Node Status: | | | | | | | 0.470 |
| Positive | 117 | (40.3%) | 49 | (41.9%) | 68 | (58,1%) | |
| Negative | 173 | (59.7%) | 80 | (46.2%) | 93 | (53.8%) | |
| MMR Status**: | | | | | | | 0.669 |
| Deficient | 25 | (78.1%) | 15 | (60.0%) | 10 | (40.0%) | |
| Proficient | 7 | (21.9%) | 3 | (42.9%) | 4 | (57.1%) | |

| | |
|---|---|
| Unknown | 266 |

¹Totals do not add to 298 patients due to missing clinical data tor several patients. Fifteen patients received neoadjuvant therapy prior to surgical resection.
²p-value while analyzing dichotomized FAK expression (“high” and “low”) using Fisher's exact test.
*p < 0.05
**MMR was no uniformly tested in the initial cohort

Our validation cohort was comprised of 517 patients with Stage I and II colorectal cancer. Of the 185 Stage I patients in this cohort, 39 (21.1%) had high FAK (Table 1). Like the initial cohort, the levels of FAK expression were significantly lower in T1 patients (p=0.047) and Stage I vs. Stage II (p=0.023, Table 3). Together these 2 cohorts showed that FAK is expressed at high levels in a subset of early stage CRC patients.

TABLE 3
Clinicopathologic Characteristics and FAK Expression, Validation cohort

| Primary Tumor | | | Low FAK | | High FAK | | |
|---|---|---|---|---|---|---|---|
| Clinicopathologic | Patients | | (Score 0 to <2) | | (Score 2 to 3) | | |
| Characteristic | (N, %)¹ | | (N, %) | | (N, %) | | P value² |
| Age | | | | | | | 1.000 |
| <70 | 255 | (49.3%) | 186 | (72.9%) | 69 | (27.1%) | |
| >70 | 262 | (50.7%) | 191 | (72.9%) | 71 | (27.1%) | |
| Gender | | | | | | | 0.692 |
| Female | 249 | (48.2%) | 184 | (73.9%) | 65 | (26.1%) | |
| Male | 268 | (51.8%) | 193 | (72.0%) | 75 | (28.0%) | |
| Stage: | | | | | | | 0.023* |
| I | 185 | (35.8%) | 146 | (78.9%) | 39 | (21.1%) | |
| II | 332 | (64.2%) | 231 | (69.6%) | 101 | (30.4%) | |
| T-stage | | | | | | | 0.047* |
| 1 | 40 | (7.7%) | 35 | (87.5%) | 5 | (12.5%) | |
| 2 | 145 | (28.0%) | 111 | (76.6%) | 34 | (23.4%) | |
| 3 | 322 | (62.3%) | 223 | (69.3%) | 99 | (30.7%) | |
| 4 | 10 | (1.9%) | 8 | (80.0%) | 2 | (20.0%) | |
| Lymphovascular | | | | | | | 0.109 |
| Invasion: | | | | | | | |
| Yes | 44 | (8.6%) | 37 | (84.1%) | 7 | (15.9%) | |
| No | 466 | (91.4%) | 337 | (72.3%) | 129 | (27.7%) | |
| Perineural | | | | | | | 0.807 |
| Invasion: | | | | | | | |
| Yes | 21 | (4.2%) | 15 | (71.4%) | 6 | (28.6%) | |
| No | 481 | (95.8%) | 351 | (73.0%) | 130 | (27.0%) | |
| Grade | | | | | | | 0.090 |
| Low grade | 437 | (89.9%) | 311 | (71.2%) | 120 | (28.8%) | |
| High grade | 49 | (10.1%) | 41 | (83.7%) | 8 | (16.3%) | |
| Location: | | | | | | | 0.168 |
| Rectosigmoid | 47 | (9.1%) | 30 | (63.8%) | 17 | (36.2%) | |
| Other | 469 | (90.9%) | 346 | (73.8%) | 123 | (26.2%) | |
| Node Status: | | | | | | | 1.000 |
| Positive | 0 | (0.0%) | 0 | (0.0%) | 0 | (0.0%) | |
| Negative | 517 | (100.0%) | 377 | (100.0%) | 140 | (100.0%) | |
| MMR: | | | | | | | <.001* |
| Proficient | 415 | (80.3%) | 286 | (68.9%) | 129 | (31.1%) | |
| Deficient | 102 | (19.7%) | 91 | (89.2%) | 11 | (10.8%) | |

¹Totals do not add to 517 patients due to missing clinical data for several patients.
²p-value while analyzing dichotomized FAK expression (“high” and “low”) using Fisher's exact test.
*p < 0.05

Stage I CRC Patients with High FAK have Reduced Survival

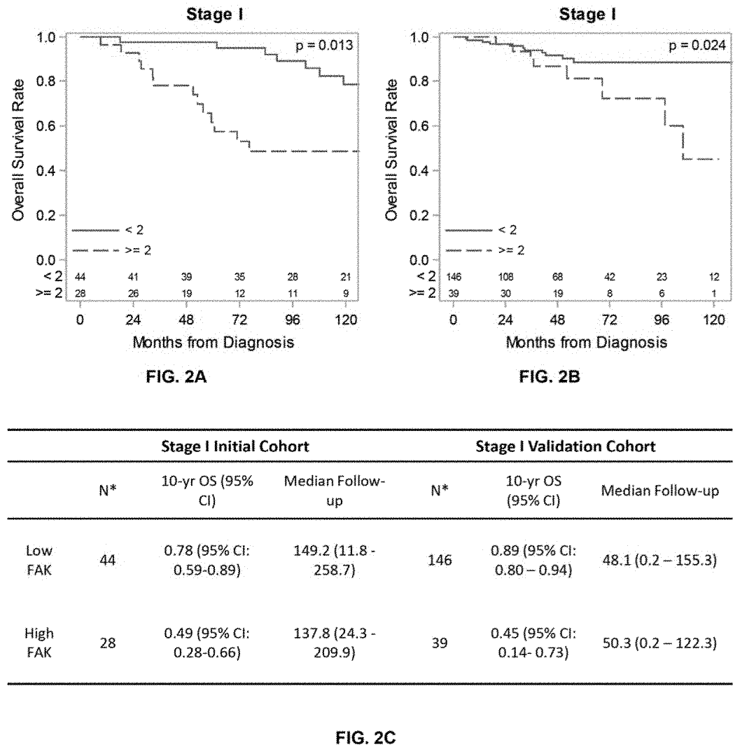

Because high levels of FAK promoted tumor invasion and metastasis, it was hypothesized that early stage tumors with high levels of FAK would be more prone to CRC dissemination than tumors with low FAK. In the initial cohort, the 10-year overall survival of the 44 Stage I patients with low FAK was 78% (95% CI 59-89%) compared to 49% (95% CI 28-66%) in the 28 patients with high FAK (p=0.013, FIGS 2A-2C), suggesting high FAK levels were associated with adverse outcome in these early stage patents. Next, an independent analysis was performed of Stage I patents in the validation cohort and found similar results. In this cohort, the 10-year overall survival of the 146 patients with low FAK was 89% (95% CI: 0.80-0.94) compared to 45% (95% CI: 0.14-0.73) in the 39 patients with high FAK (FIGS. 2A-2C). FAK expression was not statistically associated with overall survival in patients with Stage II or above tumors survival in both initial and validation cohorts (data not shown).

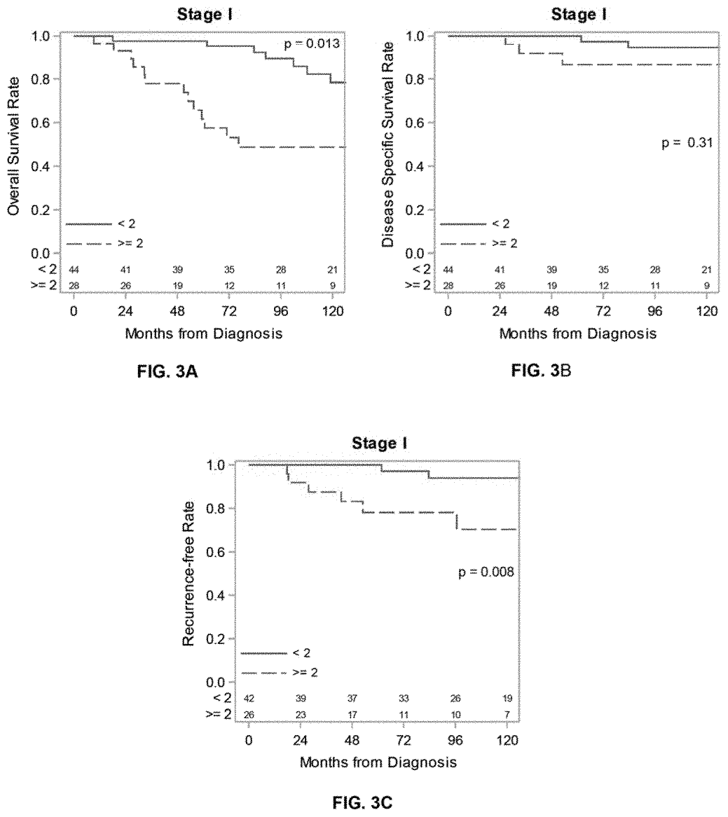

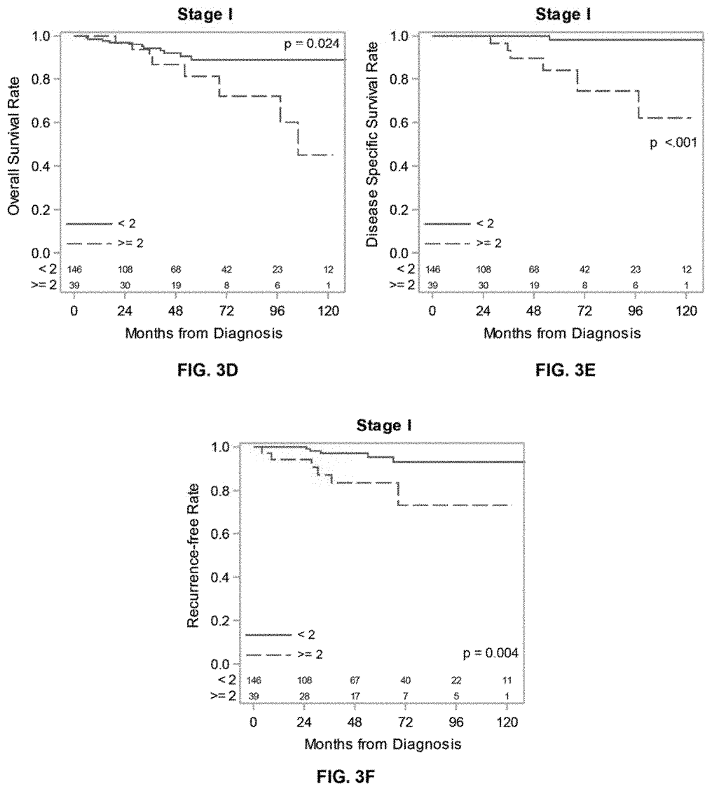

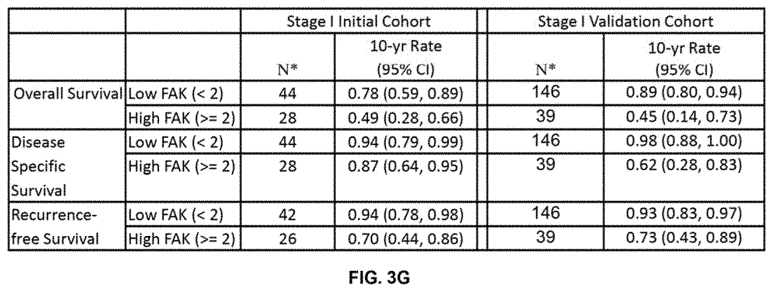

Assessment of ten-year recurrence free survivals in both initial and validation cohorts also identified significant survival differences in Stage I tumors. In the initial cohort, recurrence free survival for stage I tumors was significantly worse for high FAK tumors (70% 10-year RFS, 95% CI 44-86%), compared to low FAK tumors (94% 10-year RFS, 95% CI 78-98%, p=0.008). In the validation cohort, recurrence free survival for stage I tumors was significantly worse for high FAK tumors (73% 10-year RFS, 95% CI 43-89%), compared to low FAK tumors (93% 10-year RFS, 95% CI 83-97%, p=0.004, FIGS. 3A-3G).

Ten-year disease-specific survivals in Stage I tumors in the initial cohort did not reach significance, although it showed a trend for high FAK tumors having decreased survival (87% 10-year DSS, 95% CI 64-95%), compared to low FAK tumors (94% 10-year DSS, 95% CI 79-99%, p=0.31). However, significant disease specific survival difference was observed in the validation cohort for stage I tumors, with high FAK tumors having decreased survival (62% 10-year DSS, 95% CI 28-83%), compared to low FAK tumors (98% 10-year DSS, 95% CI 88-100%, p<0.001, FIGS. 3A-3G).

FAK Expression did not Correlate with known Adverse Histologic Factors in CRC

In the initial cohort, FAK did not correlate with any of the standard aggressive histologic features of the primary tumor, including lymph node metastasis, tumor grade and differentiation, lymphovascular invasion (LVI), or perineural invasion (PNI) (Table 4). This was also true in our validation cohort (Table 3). From these results, it was concluded that there was no existing surrogate marker of FAK on routine histopathologic analysis.

Additionally, the validation cohort was uniformly assessed for mismatch repair protein status. MMR-deficient tumors were more likely to have low FAK scores (89.2%) compared to MMR-proficient tumors (68.9%, p<0.001). However, MMR status did not predict survival among the entire cohort (log-rank p=0.772 for OS, p=for RFS), in stage I only (p=0.825 for OS, p=for RFS), or in stage II only (p=0.634 for OS, p=for RFS). This also suggests that MMR is also not a surrogate marker for FAK.

TABLE 4
Multivariate Survival Analysis for Combined Stage I Data

| Model | Variable | | Hazard Ratio | | P-value |
|---|---|---|---|---|---|
| OS | Primary Site | Colon vs Rectum | 1.83 | (0.83, 4.01) | 0.133 |
| | Lymph Invasion | Yes vs No | 3.44 | (1.15, 10.23) | 0.027* |
| | Perineural | Yes vs No | 3.10 | (0.54, 17.77) | 0.205 |
| | FAK | >=2 vs <2 | 3.27 | (1.65, 6.49) | <.001* |
| DSS | Primary Site | Colon vs Rectum | 0.44 | (0.11, 1.70) | 0.234 |
| | Lymph Invasion | Yes vs No | 15.89 | (2.28, 110.76) | 0.005* |
| | Perineural | Yes vs No | 8.36 | (0.25, 279.21) | 0.236 |
| | FAK | >=2 vs <2 | 8.42 | (1.80, 39.38) | 0.007* |
| RFS | Primary Site | Colon vs Rectum | 0.49 | (0.16, 1.48) | 0.203 |
| | Lymph Invasion | Yes vs No | 7.16 | (1.40, 36.52) | 0.018* |
| | Perineural | Yes vs No | 10.95 | (1.52, 78.87) | 0.018* |
| | FAK | >=2 vs <2 | 6.30 | (2.02, 19.69) | 0.002* |

### FAK is the Most Significant Predictor of Adverse Outcome in Multivariate Analysis

Multivariate analysis was subsequently performed on stage I patients using the combined initial and validation cohort data set. Multivariate survival analysis for stage l patients showed that the only two significant factors predicting OS were FAK expression (hazard ratio [HR] 3.27, 95% CI 1.65-6.49, p<0.001) and lymphovascular invasion (LVI), (HR 3.44, 95% CI 1.15-10.23, p=0.027). The same two factors remained to predict DSS: FAK expression (HR 8.42, 95% CI 1.80-39.38, p=0.007) and LVI (HR 15.89, 95% CI 2.28-110.76, p=0.005). Similarly, on multivariate analysis, three factors predicted RFS in stage I patients: FAK expression (HR 6.30, 95% CI 2.02-19.69, p=0.002), LVI (HR 7.16, 95% CI 1.40-36.52, p=0.018), and perineural invasion (HR 10.95, 95% CI 1.52-76.887, p=0.018). Thus, high FAK is one of the most significant predictors of poor outcome among Stage I patients (Table 4).

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings.

1. A method for preparing a tissue sample from a subject afflicted with Stage I colorectal cancer (CRC), comprising:
a) obtaining a tissue sample from the subject with Stage I CRC;
b) separating tissue samples obtained in (a) by;
i) measuring a focal adhesion kinase (FAK) protein expression in the tissue sample using immunohistochemical staining and;
ii) classifying a FAK clinical score of FAK protein expression based on the level of immunohistochemical staining measured in (b), wherein no FAK staining comprises no detectable FAK protein expression and clinical score of 0, weak FAK staining comprises low FAK protein expression and clinical score of 1, moderate FAK staining comprises moderate FAK protein expression and clinical score of 2, and strong FAK staining comprises high FAK expression and clinical score of 3 and;
c) analysing the FAK clinical score of FAK protein expression found in (b).

2. The method of claim 1, wherein the tissue sample comprises biopsy tissue, surgical resection tissue, tumor tissue, benign tissue, normal tissue, fibrotic tissue, and/or tissue culture.

3. The method of claim 1, wherein the tissue sample comprises formalin-fixed, paraffin-embedded tissue or frozen tissue.

4. The method of claim 1, wherein FAK protein expression in the tissue sample is furthered measured by standard technologies for protein, RNA, and DNA analyses comprising immunofluorescence, Western blot, ELISA, whole transcriptome sequencing, and/or measurement of phosphorylation status of tyrosine residues on FAK.

5. The method of claim 1, further comprises measuring FAK mRNA levels and/or FAK activity levels.

6. The method of claim 1, wherein the method is for further assessing FAK expression in a blood sample, a cell sample, and/or tissue culture.

7. The method of claim 6, wherein the cell sample comprises cell culture or circulating tumor cells.

8. The method of claim 6, wherein the blood sample comprises, serum, plasma, peripheral blood mononuclear cells, circulating tumor cells, circulating tumor DNA, circulating free DNA, circulating microRNA.

9. A method for personalized treatment of a subject afflicted with Stage I colorectal cancer (CRC), the method comprising the steps of:
a) determining whether the subject has an overexpression of focal adhesion kinase (FAK) by:
i) obtaining a tissue sample from the subject, wherein the tissue sample is amenable to measure FAK protein expression;
ii) measuring the FAK protein expression in the sample using immunohistochemical staining; and
iii) classifying a FAK clinical score of FAK protein expression based on the level of immunohistochemical staining measured in (b), wherein no FAK staining comprises no detectable FAK protein expression and clinical score of 0, weak FAK staining comprises low FAK protein expression and clinical score of 1, moderate FAK staining comprises moderate FAK protein expression and clinical score of 2, and strong FAK staining comprises high FAK expression and clinical score of 3; and
b) administering a therapeutically effective agent or other intervention(s) to treat the Stage I CRC based on the disease profile classified in (iii), wherein those subjects with clinical scores of 0 or 1 with low risk of recurrence would not be administered a therapeutic agent or intervention and those subjects with clinical scores of 2 or 3 with higher risk of recurrence would be administered a therapeutic drug or intervention to treat the stage I CRC.

10. The method of claim 9, wherein the tissue sample comprises biopsy tissue, surgical resection tissue, tumor tissue, benign tissue, normal tissue, fibrotic tissue, and/or tissue culture.

11. The method of claim 9, wherein the tissue sample comprises formalin-fixed, paraffin-embedded tissue or frozen tissue.

12. The method of claim 9, wherein FAK protein expression in the tissue sample is furthered measured by standard technologies for protein, RNA, and DNA analyses comprising immunofluorescence, Western blot, ELISA, whole transcriptome sequencing, and/or measurement of phosphorylation status of tyrosine residues on FAK.

13. The method of claim 9, further comprising measuring FAK mRNA levels and/or FAK activity levels.

14. The method of claim 9, wherein FAK expression is measured longitudinally.

15. The method of claim 14, wherein FAK expression measured at various times, longitudinally, throughout progression of the condition, wherein the time is at: 1) at time of diagnosis; 2) two to seven days post-diagnosis, 3) one month post-diagnosis, 4) three months post-diagnosis, or 5) >three months post-diagnosis.

16. The method of claim 9, wherein the therapeutic agent or intervention (s) comprise: inhibitors to the FAK pathway, which result in decrease production, expression, and/or activity of FAK; antibody therapy; vaccine therapy; adjuvant radiation therapy; adjuvant chemotherapy; adjuvant immunotherapy; aggressive resection surgery; and/or screening tests for CRC comprising serum carcinoembryonic antigen (CEA) levels, computed tomography (CT) scans, magnetic resonance imaging (MRI) scans, and/or positron-emission tomography-computed tomography (PET-CT) scans.

17. The method of claim 9, wherein the therapeutic agents comprises antibodies, vaccines, small molecules, cytotoxic agents comprising 5-fluorouracil, leucovorin, oxaliplatin (FOLFOX), tyrosine kinase inhibitors including both small molecules and antibody-based inhibitors, and/or immunotherapy.

18. The method of claim 9, wherein the method is for further assessing FAK expression in a blood sample, a cell sample, and/or tissue culture.

19. The method of claim 18, wherein the cell sample comprises cell culture or circulating tumor cells.

20. The method of claim 19, wherein the blood sample comprises, serum, plasma, peripheral blood mononuclear cells, circulating tumor cells, circulating tumor DNA, circulating free DNA, circulating microRNA.

21.-30. (canceled)
